(19) United States
(12) Patent Application Publication
(10) Pub. No.: US 2022/0273345 A1
(43) Pub. Date: Sep. 1, 2022

(54) AN INTRAMEDULLARY AFFIXING APPARATUS

(71) Applicants: Yevgeny KONSTANTINOVSKY, Ashdod (IL); Amit Eitan GADASI, Ramat Gan (IL)

(72) Inventor: Yevgeny KONSTANTINOVSKY, Ashdod (IL)

(21) Appl. No.: 17/632,600

(22) PCT Filed: Aug. 6, 2020
(86) PCT No.: PCT/IL2020/050860

§ 371 Date: Feb. 3, 2022

(30) Foreign Application Priority Data
Aug. 8, 2019 (IL) 268608

(51) Int. Cl. A61B 17/72; F16C 1/10; F16C 1/14

(52) U.S. Cl. CPC A61B 17/7266; F16C 1/101; F16C 1/145

(57) ABSTRACT

An intramedullary affixing apparatus, comprising: a hollow shaft suitable for insertion into a bone and adapted to accommodate a proximal anchoring device, a transmission arrangement, and a distal anchoring device, wherein the hollow shaft comprises a proximal threaded actuator and a distal threaded actuator, wherein the threading-in of the proximal threaded actuator exerts a linear motion of the proximal anchoring device, the transmission arrangement, and the distal anchoring device towards the distal edge of the hollow shaft and the threading-in of the distal actuator exerts an opposite motion.

### FIELD OF THE INVENTION

The present invention is in the field of bone healing. More particularly, the invention relates to an intramedullary affixing apparatus which can be applied in straight and in curved bones. Furthermore, the present invention can be utilized for the transfer of curved linear motion, in application of different fields.

### BACKGROUND OF THE INVENTION

Bone healing, or fracture healing, is a proliferated physiological process in which the body facilitates the repair of a bone fracture. Generally, bone fracture treatment consists of a doctor reducing (pushing) displaced bone fragments back into place via reduction, with or without anesthetic, stabilizing their position, and then waiting for the bone's natural healing process to occur.

Some of the more complex bone fractures, and medical situations resulting with compelled bone tissue removal, require an internal affixing of the affected bone involving the insertion of a an affixing rod into the bone, followed by the fixation of the rod into the bone by using prongs, nails or screws which are externally threaded through the bone and the rod, however, the prongs, nails or screws must be aimed to the exact fixation drills in the affixing rod, while the rod is hidden inside the bone, hence even with the most advanced imaging equipment at reach, the prior art affixing methods, using external affixing fasteners, are difficult for implementation.

Furthermore, many affixing rods of the prior art are straight, while many of the longer bones (i.e., which are more prone to complex fractures) are curved, resulting with a difficult insertion and positioning of the available affixing rods inside the curved bone and potentially causing damage to the internal bone tissue, thus being inhibitive to the healing of the fractured bone.

It is an object of the present invention to provide an intramedullary affixing apparatus which is simple to assemble and apply.

It is another object of the present invention to provide an intramedullary affixing apparatus which is easy to apply into curved bones.

Other objects and advantages of the invention will become apparent as the description proceeds.

### SUMMARY OF THE INVENTION

An intramedullary affixing apparatus, comprising: a hollow shaft suitable for insertion into a bone and adapted to accommodate a proximal anchoring device, a transmission arrangement, and a distal anchoring device, wherein said hollow shaft comprises a proximal threaded actuator and a distal threaded actuator, wherein the threading-in of said proximal actuator exerts a linear motion of said proximal anchoring device, transmission arrangement, and distal anchoring device towards the distal edge of said hollow shaft and the threading-in of said distal actuator exerts an opposite motion.

According to an embodiment of the invention, each of the proximal and distal anchoring devices comprises a substantially hollow piston adapted to accommodate two crankshafts and two anchoring elements, wherein a linear motion of said hollow piston with respect to said hollow shaft towards its distal edge, results with said crankshafts pushing said anchoring elements to extend through corresponding openings and stab the surrounding bone tissue and an opposite motion of said hollow piston results with the retrieval of said anchoring elements into said hollow piston.

According to an embodiment of the invention, the transmission arrangement comprises a chain of two or more drilled balls and a connecting wire having its proximal edge connected to said proximal anchoring device and its distal edge connected to the distal anchoring device, wherein said transmission arrangement enables the transfer of the linear motion of one anchoring device to the second through a defined course within said hollow shaft.

According to an embodiment of the invention, the hollow shaft is curved.

According to an embodiment of the invention, the hollow shaft is straight.

According to an embodiment of the invention, all the comprising components are constructed of titanium.

According to an embodiment of the invention, the partially curved hollow shaft is split into two halves, thereby enabling an effortless assembly of said apparatus.

According to an embodiment of the invention, the hollow pistons of the anchoring devices are split into two halves, thereby enabling an effortless assembly of said anchoring devices.

According to an embodiment of the invention, the anchoring elements are curved and pointed.

According to an embodiment of the invention, the distal edge of said hollow shaft comprises a stabilizing spring.

According to an embodiment of the invention, provided with additional threads to enable further threading of the proximal threaded actuator while the anchoring elements are fully extended, thereby providing a geometric lock of said anchoring elements in its extended position.

According to an embodiment of the invention, the threaded actuators are Allen bolts.

According to an embodiment of the invention, the transmission arrangement comprises four equally sized drilled balls.

According to an embodiment of the invention, the transmission arrangement comprises drilled balls of different diameter, thus enabling different timing synchronizations between the proximal and distal anchoring elements extension.

According to an embodiment of the invention, the substantially hollow piston is provided with an inclined wall facing the adjacent threaded actuator, thereby providing a finer control of the extension of the anchoring elements.

According to an embodiment of the invention, the substantially hollow piston comprises travel limiting grooves.

### BRIEF DESCRIPTION OF THE DRAWINGS

FIG. 1 schematically illustrates an external perspective view of an intramedullary affixing apparatus 100, according to an embodiment of the present invention;

FIG. 2 schematically illustrates an internal view of an intramedullary affixing apparatus 200 with hollow shaft 101 of FIG. 1 removed, according to an embodiment of the present invention; and

FIGS. 3A and 3B schematically illustrate enlarged internal views of the anchoring mechanism of anchoring device 220, according to an embodiment of the present invention.

### DETAILED DESCRIPTION OF THE DRAWINGS

The present invention relates to an intramedullary affixing apparatus having internal fixation means, which reduce the installation effort and eliminate further mechanical operations such as drilling and threading external fixing bolts through the bone, hence reduce the lateral damage to the bone and surrounding tissues, resulting with a shorter healing process.

The proposed apparatus is comprised of a hollow shaft which is adapted with anchoring extension orifices and with internal threads at its proximal and distal ends, and comprises proximal and distal anchoring devices in a close relationship through a mediating transmission arrangement, wherein the threading in of a proximal actuator exerts a linear motion to the proximal anchoring device towards the distal edge of the hollow shaft. The linear motion is transferred by the transmission arrangement to the distal anchoring device, which moves towards the distal actuator to an extent, which can be desirably limited by a preliminary threading in of the distal actuator. The proximal and distal anchoring devices are adapted to extend anchoring elements when forced to travel towards the distal end of the hollow shaft, and to retrieve the anchoring elements when forced to travel towards the proximal end, hence, following the insertion of the proposed apparatus into a bone, the fixation operation is simply performed from the proximal end of the proposed apparatus (i.e., and bone) and the retrieval (i.e., when the bone affixing is no longer required) is performed by releasing the proximal actuator and threading in the distal actuator (i.e., from both ends of the bone).

Reference will now be made to several embodiments of the present invention, examples of which are illustrated in the accompanying figures for purposes of illustration only. One skilled in the art will readily recognize from the following description that alternative embodiments of the comprising elements herein may be employed without departing from the principles of claimed invention.

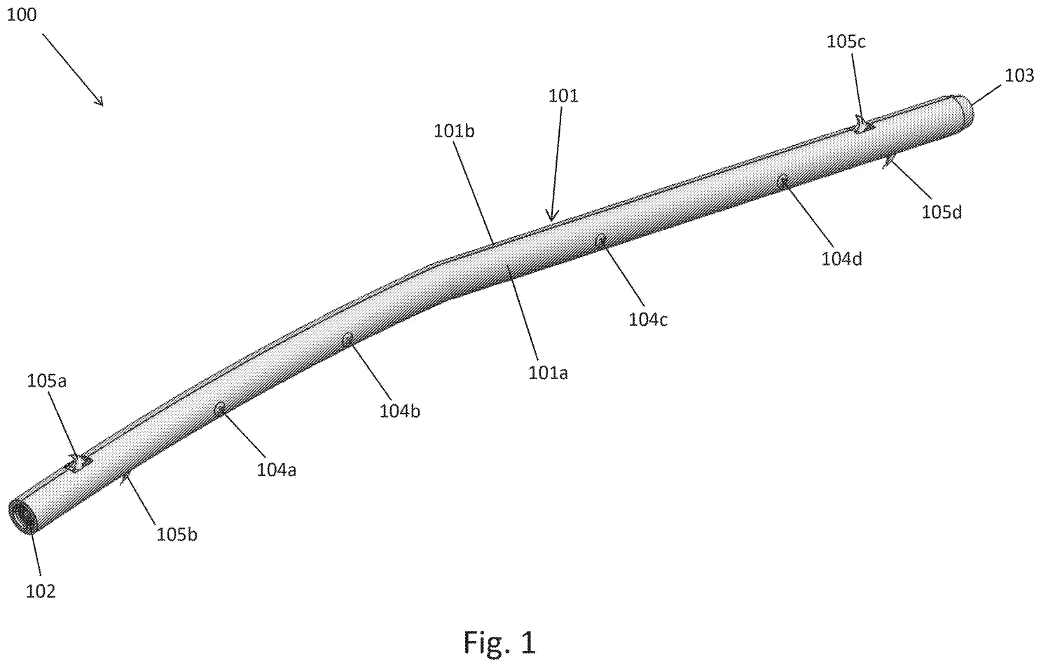

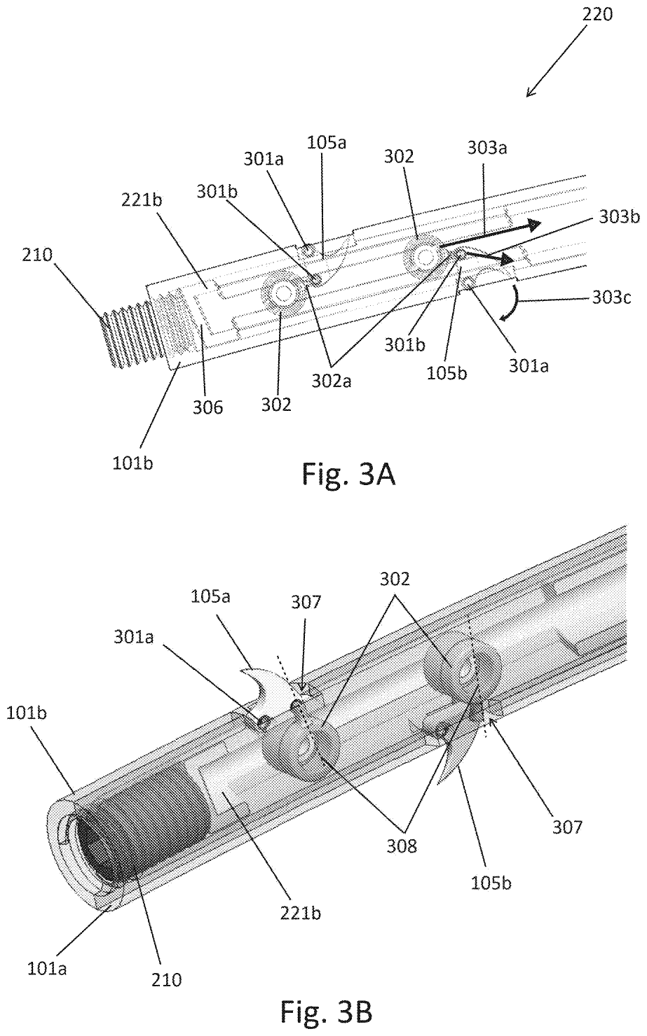

FIG. 1 schematically illustrates an external perspective view of an intramedullary affixing apparatus 100, according to an embodiment of the present invention, in which apparatus 100 comprises a hollow shaft 101 which is split into front and rear hollow shaft sections 101a and 101b, having a proximal end 102 and a distal end 103 and being fastened by fasteners 104 (e.g., countersunk bolts). Hollow shaft 101 is adapted with anchoring orifices to enable the extension of anchoring elements 105a-105d for fixing apparatus 100 inside a bone. The curved and pointed anchoring elements 105a-105d provide a firm grasp of the anchoring point at the beginning of the anchoring operation, and requires reduced retrieval effort, when the retrieval is performed in an opposite circular operation to the circular anchoring operation (further illustrated in FIGS. 3A and 3B). According to an embodiment of the present invention, the anchoring elements are of a biomimicry design in a similar shape to the claw of a predator animal.

According to some embodiments of the present invention, the proposed apparatus is partially or fully curved. A transmission arrangement, which is provided with the proposed apparatus, is capable of transferring both straight and curved linear motion, thereby enabling the internal control of the abovementioned proximal and distal anchoring means of straight and curved implementation of apparatus 100.

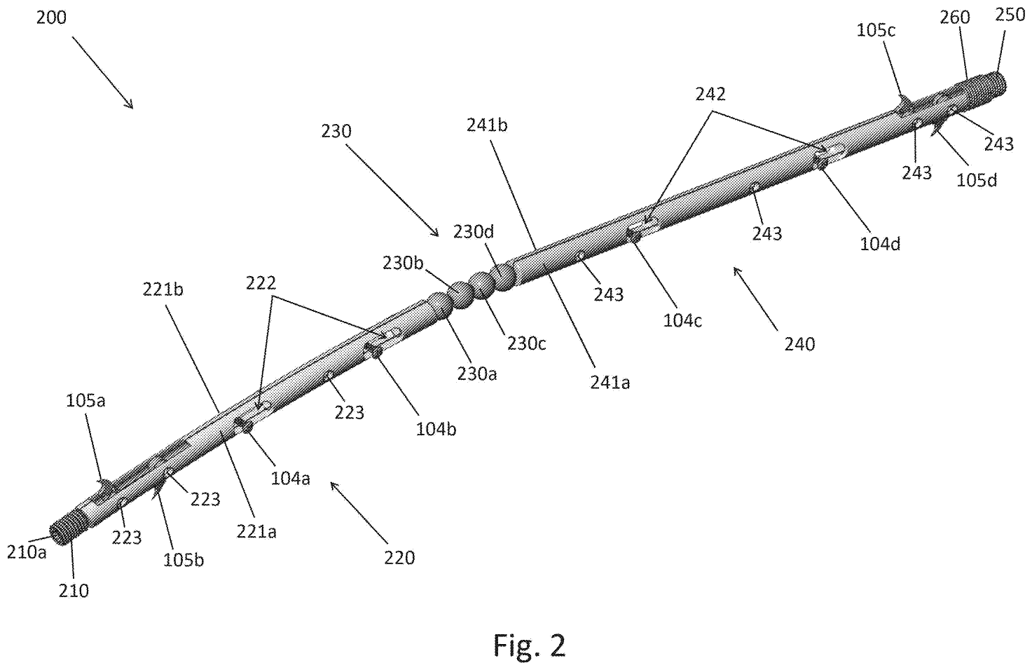

FIG. 2 schematically illustrates an internal view of an intramedullary affixing apparatus 200 with removed hollow shaft sections 101a and 101b of FIG. 1, according to an embodiment of the present invention, in which apparatus 200 comprises a proximal threaded actuator 210 with suitably socketed head 210a (e.g., Allen socket), a proximal anchoring device 220 comprising front and rear hollow piston sections 221a and 221b with attachment drills 223 thereof (i.e., attachment fasteners are not shown, yet a skilled person in the art will realize multiple available attachment arrangements such as a sunk head bolt threaded through section 221a into a corresponding threading socket in section 221b), a transmission arrangement 230, a distal anchoring device 240 comprising front and rear hollow piston sections 241a and 241b with attachment drills 243 thereof (i.e., similarly with drills 223), and a distal threaded actuator 250 with suitably socketed head 210a (not shown), wherein proximal actuator 210 is threaded in, hence, proximal and distal anchoring devices 220 and 240 are at their distal position, and anchoring elements 105a-105d at their extended state.

A skilled person in the art will readily observe the curvature of proximal anchoring device 220 and the non-flat angle between devices 220 and 240, yet the linear motion of proximal anchoring device 220 is transferred by the transmission arrangement 230 to distal anchoring device 240, which moves towards the distal actuator 250. The smooth transfer of the linear motion inside the curved hollow shaft 101 (of FIG. 1) is enabled by the chain of drilled balls 230a-230d, each of which can limitedly roll with respect to each other, while being connected through with an internal connecting wire (not shown) which is threaded through balls 230a-230d and connects to proximal and distal anchoring devices 220 and 240 at its both ends. Of course, multiple designs of transmission arrangement 230 comprising chained elements of different quantities, sizes and shapes (i.e., which can limitedly move with respect to each other, thus transfer a curved linear motion) and differently interconnections (e.g., titanium wiring bundle) can be selected by the skilled person in the art, in accordance with specific applications of the proposed affixing apparatus. For example, having one or more balls 230a-230d with different diameter will provide different transmission ratio, resulting with different extension timing of distal anchoring elements 105c and 105d, with respect to elements 105a and 105b, which can be desired in some cases such as when a physician would like to initially extend the proximal anchoring elements 105a and 105b, then to verify the position of distal end 103 of FIG. 1 and only then to further thread in proximal threaded actuator 210, thus to extend the distal anchoring elements 105c and 105d.

According to some embodiments, the comprising elements of the proposed apparatus are made of metals and particularly titanium alloys, of which presence in a living body is proven as safe.

Further shown in FIG. 2 are guiding grooves 222 and 242 of devices 220 and 240, which are utilized in conjunction with fasteners 104 (i.e., which are threaded through hollow shaft 101 (of FIG. 1) and through in grooves 222 and 242), to define the extent to which devices 220 and 240 can travel inside hollow shaft 101. Furthermore, the extent to which devices 220 and 240 travel inside hollow shaft 101, can desirably limited by a preliminary threading in of distal actuator 250. According to an embodiment of the present invention, apparatus 200 further comprises a resisting spring 260 which provides a desirable anchoring resistance (i.e., added resistance which provides a finer ratio between the turning force of proximal threaded actuator 210 and the induced extension of anchoring elements 105a-105d) and residual load stored in spring 260 which can assist with the initial retraction of anchoring elements 105a-105d when proximal threaded actuator 210 is released.

FIGS. 3A and 3B schematically illustrate enlarged internal views of the anchoring mechanism of anchoring device 220, according to an embodiment of the present invention, where FIG. 3A illustrates anchoring elements 105a and 105b in a retrieved state within anchoring device 220, each of which is stationary hinged to shaft 101 at one vertex 301a and movably hinged to arm 302a of crankshaft 302 at second vertex 301b, wherein crankshaft 302 is stationary hinged to hollow piston 221 (i.e., for the sake of clarity, only the rear sections 101b and 221b of hollow shaft 101 and hollow piston 221 are shown in FIGS. 3A and 3b, yet the hinging of anchoring elements 105a and 105b to hollow shaft 101 is supported by both rear and front hollow shaft sections 101a and 101b, and the hinging of crankshaft 302 to hollow piston 221 is supported by both rear and front piston sections 221a and 221b), thereby when piston 221 is forced (i.e., by the threading in of actuator 210 which pushes wall 306 of piston 221) to travel towards the distal end 103 as illustrated by arrow 303a of FIG. 3A, arms 302a of crankshafts 302 thrust vertices 301b, thus rotating anchoring elements 105a and 105b (i.e., around hinged vertices 301a) to extend outside of piston 220 as indicated by arrow 303c. The release of proximal actuator 210, followed by the threading in of distal actuator 250 (shown in FIG. 2), results with the retrieval of anchoring elements 105a and 105b into piston 220.

According to an embodiment of the present invention, piston 220 is provided with an inclined wall 306, thereby increasing the ration between the threading of actuator 210 and the exerted travel of piston 220, hence providing a finer control of the extension of anchoring elements 105a-105d.

The abovementioned mechanism which converts the linear motion of piston 220 to a circular extension of elements 105a and 105b is similarly utilized at the distal anchoring device 240 and the extension/retrieval of anchoring elements 105c and 105d. According to some embodiments of the present invention, the anchoring mechanisms of devices 220 and 240 are of the same design. According to other embodiments, devices 220 and 240 or some of their comprising elements can be differently designed, in accordance with specific geometrical conditions, desirable assembly process flow or assembly faults prevention. For example, crankshafts 302 of anchoring device 220 may be differently sized than crankshafts 302 of anchoring device 240, thereby obstructing the assembly of differently sized crankshafts 302 to the wrong anchoring device.

FIG. 3B illustrates anchoring elements 105a and 105b in an extended state, where according to some embodiments of the present invention, shaft 101 is provided with additional proximal threads to enable further threading of proximal actuator 210 while anchoring elements 105a-105d are fully extended, forcing a further travel of crankshaft 302 towards the distal end 103 (shown in FIG. 1), resulting with a geometric lock of anchoring elements 105a-105d in its extended position, as indicated by dashed lines 308. The geometric lock provides an improved anchoring which is less susceptible to vibrations and undesirable mechanical shocks induced on the healing bone.

Although embodiments of the invention have been described by way of illustration, it will be understood that the invention may be carried out with many variations, modifications, and adaptations, without exceeding the scope of the claims. Furthermore, one skilled in the art will readily observe that the proposed apparatus and its comprising elements may be provided in different sizes, and geometries, suitable for multiple different applications (e.g., different bone shapes and dimensions), without departing the principals of the present invention.

1. An intramedullary affixing apparatus, comprising: a hollow shaft suitable for insertion into a bone and adapted to accommodate a proximal anchoring device, a transmission arrangement, and a distal anchoring device, wherein said hollow shaft comprises a proximal threaded actuator and a distal threaded actuator, wherein the threading-in of said proximal actuator exerts a linear motion of said proximal anchoring device, transmission arrangement, and distal anchoring device towards the distal edge of said hollow shaft and the threading-in of said distal actuator exerts an opposite motion.

2. The affixing apparatus according to claim 1, wherein each anchoring device comprises a substantially hollow piston adapted to accommodate two crankshafts and two anchoring elements, wherein a linear motion of said hollow piston with respect to the hollow shaft towards its distal edge, results with said crankshafts pushing said anchoring elements to extend through corresponding openings and stab the surrounding bone tissue and an opposite motion of said hollow piston results with the retrieval of said anchoring elements into said hollow piston.

3. The affixing apparatus according to claim 1, wherein the transmission arrangement comprises a chain of two or more drilled balls and a connecting wire having its proximal edge connected to the proximal anchoring device and its distal edge connected to the distal anchoring device, wherein said transmission arrangement enables the transfer of the linear motion of one anchoring device to the second through a defined course within said hollow shaft.

4. The affixing apparatus of claim 1, in which the hollow shaft is curved.

5. The affixing apparatus of claim 1, in which the hollow shaft is straight.

6. The affixing apparatus of claim 1, in which all the comprising components are constructed of metals.

7. The affixing apparatus of claim 1, in which the partially curved hollow shaft is split into two halves, thereby enabling an effortless assembly of said apparatus.

8. The affixing apparatus of claim 1, in which the hollow pistons of the anchoring devices are split into two halves, thereby enabling an effortless assembly of said anchoring devices.

9. The affixing apparatus of claim 1, in which the proximal and distal anchoring devices are of the same design.

10. The affixing apparatus of claim 1, in which the proximal and distal anchoring devices are of different designs.

11. The affixing apparatus of claim 2, in which the anchoring elements are curved and pointed.

12. The affixing apparatus of claim 11, in which the anchoring elements are of a biomimicry design similar to a predator's claw.

13. The affixing apparatus of claim 1, in which the distal edge of said hollow shaft comprises a stabilizing spring.

14. The affixing apparatus of claim 1, in which the distal threaded actuator is preliminary threaded in, thus limiting the travel of the anchoring device, hence the extension of the anchoring elements.

15. The affixing apparatus of claim 1, wherein said apparatus is provided with additional threads to enable further threading of the proximal threaded actuator while the anchoring elements are fully extended, thereby providing a geometric lock of said anchoring elements in its extended position.

16. The affixing apparatus of claim 1, in which the threaded actuators are Allen bolts.

17. The affixing apparatus of claim 3, in which the transmission arrangement comprises four equally sized drilled balls.

18. The affixing apparatus of claim 3, in which the transmission arrangement comprises drilled balls of different diameter, thus enabling different timing synchronizations between the proximal and distal anchoring elements extension.

19. The affixing apparatus of claim 2, in which the substantially hollow piston is provided with an inclined wall facing the adjacent threaded actuator, thereby providing a finer control of the extension of the anchoring elements.

20. The affixing apparatus of claim 2, in which the substantially hollow piston comprises travel limiting grooves.

21. The affixing apparatus of claim 6, in which all the comprising components are constructed of titanium.
